(19) United States
(12) Patent Application Publication
(10) Pub. No.: US 2021/0369835 A1
(43) Pub. Date: Dec. 2, 2021

(54) Method of Vaccination for SARS Virus

(71) Applicant: Shawne Forrest, Dallas, TX (US)

(72) Inventor: Shawne Forrest, Dallas, TX (US)

(21) Appl. No.: 17/022,475

(22) Filed: Sep. 16, 2020

(51) Int. Cl. A61K 39/215; A61K 39/145; C12N 15/113

(52) U.S. Cl. CPC A61K 39/215; A61K 39/145; A61K 2039/5254; C12N 2770/20022; C12N 2770/20034; C12N 15/1131

(57) ABSTRACT

A method of vaccination for severe acute respiratory syndrome (SARS) disease caused by at least one of SARS-CoV-1 virus and SARS-CoV-2 virus is achieved in inoculating a human with a specific vaccine composition. The vaccine composition includes a solution of salt water (H₂O and NaCl), ascorbic acid (Vitamin C) and/or a ascorbic acid salt and/or citric acid and/or a citric acid salt and a source of the at least one of SARS-CoV-1 virus and SARS-CoV-2 virus. The vaccine composition is introduced into at least one of a lymph node, a hair follicle, and an ear canal of the human for purposes of vaccination for SARS disease.

### TECHNICAL FIELD

The invention relates to methods of inoculating against certain infectious viruses.

### BACKGROUND

With the recent outbreak of the coronavirus that causes COVID-19 respiratory disease, the need for a vaccine has been of major concern. The coronavirus that causes the COVID-19 disease is the SARS-CoV-2 virus strain. Currently, there is no FDA approved vaccine in the United States for such virus. The SARS-CoV-1 virus strain also is a virus that causes severe acute respiratory syndrome (SARS) disease. The present invention provides a method of vaccination for SARS disease that is caused by one or both of the SARS-CoV-1 virus and the SARS-CoV-2 virus.

### DETAILED DESCRIPTION

A vaccine composition comprising a solution of salt water (H₂O and NaCl) with ascorbic acid (Vitamin C) and/or a ascorbic acid salt and/or citric acid and/or a citric acid salt and a source of the at least one of SARS-CoV-1 virus and SARS-CoV-2 virus. The vaccine composition can be used as a vaccine to inoculate someone from these viruses. Because chlorine is known to kill or weaken the SARS-CoV-1 virus and the SARS-CoV-2 virus, administering a composition containing the SARS-CoV-1 virus and/or the SARS-CoV-2 virus and chlorine to a person can help boost the person's immunity without promoting SARS disease.

The salt-water solution (H₂O+NaCl) may be that which is hypotonic (i.e., above 0.9 wt. % NaCl) in the vaccine composition. In certain embodiments, the salt-water solution may have a sodium chloride concentration of from at least, equal to, and/or between any two of 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2.0 wt. %, 2.1 wt. %, 2.2 wt. %, 2.3 wt. %, 2.4 wt. %, 2.5 wt. %, 2.6 wt. %, 2.7 wt. %, 2.8 wt. %, 2.9 wt. %, 3.0 wt. %, 3.1 wt. %, 3.2 wt. %, 3.3 wt. %, 3.4 wt. %, 3.5 wt. %, 3.6 wt. %, 3.7 wt. %, 3.8 wt. %, 3.9 wt. %, 4.0 wt. %, 4.1 wt. %, 4.2 wt. %, 4.3 wt. %, 4.4 wt. %, 4.5 wt. %, 4.6 wt. %, 4.7 wt. %, 4.8 wt. %, 4.9 wt. %, 5.0 wt. %, 5.5 wt. %, 6.0 wt. %, 6.5 wt. %, 7.5 wt. %, 8.0 wt. %, 8.5 wt. %, 9.0 wt. %, 9.5 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, and 28 wt. % by total weight of the salt-water solution. The sodium chloride concentration in the vaccine composition should be sufficiently high to ensure that the SARS-CoV-1 and/or SARS-CoV-2 viruses are sufficiently weakened so they do not promote SARS disease.

It should be noted in the description, if a numerical value, concentration or range is presented, each numerical value should be read once as modified by the term “about” (unless already expressly so modified), and then read again as not so modified unless otherwise indicated in context. Also, in the description, it should be understood that an amount range listed or described as being useful, suitable, or the like, is intended that any and every value within the range, including the end points, is to be considered as having been stated. For example, “a range of from 1 to 10” is to be read as indicating each and every possible number along the continuum between about 1 and about 10. Thus, even if specific points within the range, or even no point within the range, are explicitly identified or referred to, it is to be understood that the inventor appreciates and understands that any and all points within the range are to be considered to have been specified, and that inventor possesses the entire range and all points within the range, including smaller ranges within the larger ranges.

The salt-water solution may be used in the vaccine composition in an amount of from at least, equal to, and/or between any two of 20 wt. %, 21 wt. %, 22 wt. %, 23 wt. %, 24 wt. %, 25 wt. %, 26 wt. %, 27 wt. %, 28 wt. %, 29 wt. %, 30 wt. %, 31 wt. %, 32 wt. %, 33 wt. %, 34 wt. %, 35 wt. %, 36 wt. %, 37 wt. %, 38 wt. %, 39 wt. %, 40 wt. %, 41 wt. %, 42 wt. %, 43 wt. %, 44 wt. %, 45 wt. %, 46 wt. %, 47 wt. %, 48 wt. %, 49 wt. %, 50 wt. %, 51 wt. %, 52 wt. %, 53 wt. %, 54 wt. %, 55 wt. %, 56 wt. %, 57 wt. %, 58 wt. %, 59 wt. %, 60 wt. % by total weight of the vaccine composition.

The ascorbic acid and/or ascorbic acid salt may be used in the vaccine composition in an amount of from at least, equal to, and/or between any two of 0.001 wt. %, 0.002 wt. %, 0.003 wt. %, 0.004 wt. %, 0.005 wt. %, 0.006 wt. %, 0.007 wt. %, 0.008 wt. %, 0.009 wt. %, 0.01 wt. %, 0.02 wt. %, 0.03 wt. %, 0.04 wt. %, 0.05 wt. %, 0.06 wt. %, 0.07 wt. %, 0.08 wt. %, 0.09 wt. %, 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2.0 wt. %, 2.1 wt. %, 2.2 wt. %, 2.3 wt. %, 2.4 wt. %, 2.5 wt. %, 2.6 wt. %, 2.7 wt. %, 2.8 wt. %, 2.9 wt. %, 3.0 wt. %, 3.1 wt. %, 3.2 wt. %, 3.3 wt. %, 3.4 wt. %, 3.5 wt. %, 3.6 wt. %, 3.7 wt. %, 3.8 wt. %, 3.9 wt. %, 4.0 wt. %, 4.1 wt. %, 4.2 wt. %, 4.3 wt. %, 4.4 wt. %, 4.5 wt. %, 4.6 wt. %, 4.7 wt. %, 4.8 wt. %, 4.9 wt. %, 5.0 wt. %, 5.5 wt. %, 6.0 wt. %, 6.5 wt. %, 7.5 wt. %, 8.0 wt. %, 8.5 wt. %, 9.0 wt. %, 9.5 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, 30 wt. %, 31 wt. %, 32 wt. %, 33 wt. %, 34 wt. %, 35 wt. %, 36 wt. %, 37 wt. %, 38 wt. %, 39 wt. %, 40 wt. %, 41 wt. %, 42 wt. %, 43 wt. %, 44 wt. %, 45 wt. %, 46 wt. %, 47 wt. %, 48 wt. %, 49 wt. %, 50 wt. %, 51 wt. %, 52 wt. %, 53 wt. %, 54 wt. %, 55 wt. %, 56 wt. %, 57 wt. %, 58 wt. %, 59 wt. %, 60 wt. % by total weight of the vaccine composition.

The citric acid and/or a citric acid salt component may be used in an amount of from at least, equal to, and/or between any two of 0.001 wt. %, 0.002 wt. %, 0.003 wt. %, 0.004 wt. %, 0.005 wt. %, 0.006 wt. %, 0.007 wt. %, 0.008 wt. %, 0.009 wt. %, 0.01 wt. %, 0.02 wt. %, 0.03 wt. %, 0.04 wt. %, 0.05 wt. %, 0.06 wt. %, 0.07 wt. %, 0.08 wt. %, 0.09 wt. %, 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2.0 wt. %, 2.1 wt. %, 2.2 wt. %, 2.3 wt. %, 2.4 wt. %, 2.5 wt. %, 2.6 wt. %, 2.7 wt. %, 2.8 wt. %, 2.9 wt. %, 3.0 wt. %, 3.1 wt. %, 3.2 wt. %, 3.3 wt. %, 3.4 wt. %, 3.5 wt. %, 3.6 wt. %, 3.7 wt. %, 3.8 wt. %, 3.9 wt. %, 4.0 wt. %, 4.1 wt. %, 4.2 wt. %, 4.3 wt. %, 4.4 wt. %, 4.5 wt. %, 4.6 wt. %, 4.7 wt. %, 4.8 wt. %, 4.9 wt. %, 5.0 wt. %, 5.5 wt. %, 6.0 wt. %, 6.5 wt. %, 7.5 wt. %, 8.0 wt. %, 8.5 wt. %, 9.0 wt. %, 9.5 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, 30 wt. %, 31 wt. %, 32 wt. %, 33 wt. %, 34 wt. %, 35 wt. %, 36 wt. %, 37 wt. %, 38 wt. %, 39 wt. %, 40 wt. %, 41 wt. %, 42 wt. %, 43 wt. %, 44 wt. %, 45 wt. %, 46 wt. %, 47 wt. %, 48 wt. %, 49 wt. %, 50 wt. %, 51 wt. %, 52 wt. %, 53 wt. %, 54 wt. %, 55 wt. %, 56 wt. %, 57 wt. %, 58 wt. %, 59 wt. %, and 60 wt. % by total weight of the vaccine composition.

A source of one or both of the SARS-CoV-1 virus and SARS-CoV-2 virus vaccine is also used in the vaccine composition. The source of the SARS-CoV-1 virus and/or the SARS-CoV-2 virus can be the sputum or other bodily fluid of an infected person containing the virus. In other embodiments the SARS-CoV-1 virus and/or the SARS-CoV-2 virus may be an isolated and/or purified source of the virus. The SARS-CoV-1 virus and/or SARS-CoV-2 virus component may be used in an amount of from at least, equal to, and/or between any two of 0.00001 wt. %, 0.00002 wt. %, 0.00003 wt. %, 0.00004 wt. %, 0.00005 wt. %, 0.00006 wt. %, 0.00007 wt. %, 0.00008 wt. %, 0.00009 wt. % 0.0001 wt. %, 0.0002 wt. %, 0.0003 wt. %, 0.0004 wt. %, 0.0005 wt. %, 0.0006 wt. %, 0.0007 wt. %, 0.0008 wt. %, 0.0009 wt. %, 0.001 wt. %, 0.002 wt. %, 0.003 wt. %, 0.004 wt. %, 0.005 wt. %, 0.006 wt. %, 0.007 wt. %, 0.008 wt. %, 0.009 wt. %, 0.01 wt. %, 0.02 wt. %, 0.03 wt. %, 0.04 wt. %, 0.05 wt. %, 0.06 wt. %, 0.07 wt. %, 0.08 wt. %, 0.09 wt. %, 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2.0 wt. %, 2.1 wt. %, 2.2 wt. %, 2.3 wt. %, 2.4 wt. %, 2.5 wt. %, 2.6 wt. %, 2.7 wt. %, 2.8 wt. %, 2.9 wt. %, 3.0 wt. %, 3.1 wt. %, 3.2 wt. %, 3.3 wt. %, 3.4 wt. %, 3.5 wt. %, 3.6 wt. %, 3.7 wt. %, 3.8 wt. %, 3.9 wt. %, 4.0 wt. %, 4.1 wt. %, 4.2 wt. %, 4.3 wt. %, 4.4 wt. %, 4.5 wt. %, 4.6 wt. %, 4.7 wt. %, 4.8 wt. %, 4.9 wt. %, 5.0 wt. %, 5.5 wt. %, 6.0 wt. %, 6.5 wt. %, 7.5 wt. %, 8.0 wt. %, 8.5 wt. %, 9.0 wt. %, 9.5 wt. %, 10 wt. %, 11 wt. %, 12 wt. %, 13 wt. %, 14 wt. %, 15 wt. %, 16 wt. %, 17 wt. %, 18 wt. %, 19 wt. %, and 20 wt. % by weight of vaccine composition.

In certain instances, the SARS-CoV-1 virus and/or the SARS-CoV-2 virus may each be present in the vaccine an amount of from 0.1 μg to 100 μg per dose of the vaccine composition. In certain embodiments, the SARS-CoV-1 virus and/or SARS-CoV-2 virus may be present in an amount of from at least, equal to, and/or between any two of 0.1 μg, 0.2 μg, 0.3 μg, 0.4 μg, 0.5 μg, 0.6 μg, 0.7 μg, 0.8 μg, 0.9 μg, 1.0 μg, 1.1 μg, 1.2 μg, 1.3 μg, 1.4 μg, 1.5 μg, 1.6 μg, 1.7 μg, 1.8 μg, 1.9 μg, 2.0 μg, 2.1 μg, 2.2 μg, 2.3 μg, 2.4 μg, 2.5 μg, 2.6 μg, 2.7 μg, 2.8 μg, 2.9 μg, 3.0 μg, 3.1 μg, 3.2 μg, 3.3 μg, 3.4 μg, 3.5 μg, 3.6 μg, 3.7 μg, 3.8 μg, 3.9 μg, 4.0 μg, 4.1 μg, 4.2 μg, 4.3 μg, 4.4 μg, 4.5 μg, 4.6 μg, 4.7 μg, 4.8 μg, 4.9 μg, 5.0 μg, 5.5 μg, 6.0 μg, 6.5 μg, 7.5 μg, 8.0 μg, 8.5 μg, 9.0 μg, 9.5 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, and 100 μg per dose of the vaccine composition.

These compounds can be combined in different proportions in an aqueous solution or other suitable carrier liquid or compound to form the vaccine composition. The water or carrier liquid or compound may make up from 40 wt. % to 99 wt. % of the vaccine composition, which may include the solvent used to dissolve the various compounds.

The composition can be administered by applying the composition to different areas. This can include at least one of a lymph node, a hair follicle, and the ear canal. In the case of the lymph node, this may be a lymph node located in the neck area. With respect to the hair follicle, the vaccine composition is introduced so that it interacts with the sebum of the follicle. The composition may be administered by making a small cut or scratch in these areas and applying the vaccine composition to the cut or scratch. This is similar to the manner in which the first smallpox vaccine was administered. In other instances, it may be injected into these areas with a syringe or other suitable delivery device.

In addition to the areas noted above, the vaccine composition may also be applied to the armpit area. This too may be administered by forming a small cut or scratch and applying the vaccine composition to the cut or scratch or injecting it. The armpit area where the vaccine is administered may be located on the opposite side of the body from the lymph node, hair follicle, and/or ear canal that has been treated with the vaccine composition.

The goal of administering the vaccine composition in this manner is to replicate something like the appendix which will allow prolonged exposure to the weakened virus to keep the body in a hyper immune state by maintaining low levels of the virus. This essentially modifies the individual's immune system by keeping it in a hyper immune state. There is evidence that exposure to bee venom over time can result in an immune response that protects against future venom exposure. This has been observed in certain indigenous tribes in Africa when harvesting honey wherein those harvesting honey from beehives do not suffer any harm after being stung repeatedly by honeybees. The present invention for the vaccination for SARS-CoV-1 virus and/or SARS-CoV-2 virus has similarities to this.

In certain cases, this treatment may be used in conjunction with administering of plasma containing Coronavirus antibodies from a previously infected person that has recovered from the infection prior administering the vaccine composition. This may be achieved through a transfusion of the plasma from 5 minutes to 1 hour or more prior to administering the vaccine composition. Although, the virus is weakened by the particular vaccine composition, the administering of such plasma may provide initial and/or additional protection to the individual from the weakened virus.

The inoculation with the vaccine composition, as described above, may be carried out within a day, several days, or one week or more from being infected with the at least one of SARS-CoV-1 virus and SARS-CoV-2 virus. If necessary, the inoculation may be repeated over periods of time.

It may be expected that an outbreak of the SARS-CoV-1 virus and/or SARS-CoV-2 virus may occur from every 10 to 20 years. Such a vaccine administered in healthy subjects may quickly stop the virus while providing minimal mutation factor in the immune system as it relates to vaccinations.

In certain embodiments, the vaccine composition, as described above, may further include a source of influenza A virus. This may include the influenza A virus that contains at least one or more of the antigens h1 to h8 and n1 to n4. Each influenza A virus may be may be used in an amount of from at least, equal to, and/or between any two of 0.00001 wt. %, 0.00002 wt. %, 0.00003 wt. %, 0.00004 wt. %, 0.00005 wt. %, 0.00006 wt. %, 0.00007 wt. %, 0.00008 wt. %, 0.00009 wt. % 0.0001 wt. %, 0.0002 wt. %, 0.0003 wt. %, 0.0004 wt. %, 0.0005 wt. %, 0.0006 wt. %, 0.0007 wt. %, 0.0008 wt. %, 0.0009 wt. %, 0.001 wt. %, 0.002 wt. %, 0.003 wt. %, 0.004 wt. %, 0.005 wt. %, 0.006 wt. %, 0.007 wt. %, 0.008 wt. %, 0.009 wt. %, 0.01 wt. %, 0.02 wt. %, 0.03 wt. %, 0.04 wt. %, 0.05 wt. %, 0.06 wt. %, 0.07 wt. %, 0.08 wt. %, 0.09 wt. %, 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2.0 wt. %, 2.1 wt. %, 2.2 wt. %, 2.3 wt. %, 2.4 wt. %, 2.5 wt. %, 2.6 wt. %, 2.7 wt. %, 2.8 wt. %, 2.9 wt. %, 3.0 wt. %, 3.1 wt. %, 3.2 wt. %, 3.3 wt. %, 3.4 wt. %, 3.5 wt. %, 3.6 wt. %, 3.7 wt. %, 3.8 wt. %, 3.9 wt. %, 4.0 wt. %, 4.1 wt. %, 4.2 wt. %, 4.3 wt. %, 4.4 wt. %, 4.5 wt. %, 4.6 wt. %, 4.7 wt. %, 4.8 wt. %, 4.9 wt. %, 5.0 wt. %, 5.5 wt. %, 6.0 wt. %, 6.5 wt. %, 7.5 wt. %, 8.0 wt. %, 8.5 wt. %, 9.0 wt. %, 9.5 wt. %, 10 wt. %, 11 wt. %, 12 wt. %, 13 wt. %, 14 wt. %, 15 wt. %, 16 wt. %, 17 wt. %, 18 wt. %, 19 wt. %, and 20 wt. % by weight of vaccine composition.

In certain instances, each influenza A virus may each be present in the vaccine composition an amount of from 0.1 μg to 100 μg per dose of the vaccine composition. In certain embodiments, each influenza A virus may be present in an amount of from at least, equal to, and/or between any two of 0.1 μg, 0.2 μg, 0.3 μg, 0.4 μg, 0.5 μg, 0.6 μg, 0.7 μg, 0.8 μg, 0.9 μg, 1.0 μg, 1.1 μg, 1.2 μg, 1.3 μg, 1.4 μg, 1.5 μg, 1.6 μg, 1.7 μg, 1.8 μg, 1.9 μg, 2.0 μg, 2.1 μg, 2.2 μg, 2.3 μg, 2.4 μg, 2.5 μg, 2.6 μg, 2.7 μg, 2.8 μg, 2.9 μg, 3.0 μg, 3.1 μg, 3.2 μg, 3.3 μg, 3.4 μg, 3.5 μg, 3.6 μg, 3.7 μg, 3.8 μg, 3.9 μg, 4.0 μg, 4.1 μg, 4.2 μg, 4.3 μg, 4.4 μg, 4.5 μg, 4.6 μg, 4.7 μg, 4.8 μg, 4.9 μg, 5.0 μg, 5.5 μg, 6.0 μg, 6.5 μg, 7.5 μg, 8.0 μg, 8.5 μg, 9.0 μg, 9.5 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, and 100 μg per dose of the vaccine composition.

While the invention has been shown in some of its forms, it should be apparent to those skilled in the art that it is not so limited, but is susceptible to various changes and modifications without departing from the scope of the invention. Accordingly, it is appropriate that the appended claims be construed broadly and in a manner consistent with the scope of the invention.

I claim:

1. A method of vaccination for severe acute respiratory syndrome (SARS) disease caused by at least one of SARS-CoV-1 virus and SARS-CoV-2 virus, the method comprising:
inoculating a human by introducing a vaccine composition comprising a solution of salt water (H₂O and NaCl) with ascorbic acid (Vitamin C) and/or a ascorbic acid salt and/or citric acid and/or a citric acid salt and a source of the at least one of SARS-CoV-1 virus and SARS-CoV-2 virus into at least one of a lymph node, a hair follicle, and an ear canal of the human for purposes of vaccination for SARS disease.

2. The method of claim 1, wherein:
the source of the at least one of SARS-CoV-1 virus and SARS-CoV-2 virus comprises sputum from an infected person.

3. The method of claim 1, wherein:
the lymph node is that present in the neck of the human.

4. The method of claim 1, wherein:
the vaccine composition is also introduced into a lymph node located under the armpit of the human.

5. The method of claim 1, further comprising:
administering a transfusion of blood plasma from a previously infected person to the human prior to or while introducing the vaccine composition.

6. The method of claim 5, wherein:
the transfusion is administered from 5 minutes to 1 hour prior to introducing the vaccine composition.

7. The method of claim 1, wherein:
the inoculation is carried out within one week from being infected with the at least one of SARS-CoV-1 virus and SARS-CoV-2 virus.

8. The method of claim 1, wherein:
the vaccine composition further includes a source of influenza A virus.

9. The method of claim 8, wherein:
the influenza A virus is that containing at least one or more of the antigens h1 to h8 and n1 to n4.

10. The method of claim 1, wherein:
the at least one of SARS-CoV-1 virus and SARS-CoV-2 virus is SARS-CoV-2 virus.

11. A method of vaccination for severe COVID 19 disease caused by SARS-CoV-2 virus, the method comprising:
inoculating a human by introducing a vaccine composition comprising a solution of salt water (H₂O and NaCl) with ascorbic acid (Vitamin C) and/or a ascorbic acid salt and/or citric acid and/or a citric acid salt and a source of the SARS-CoV-2 virus into at least one of a lymph node, a hair follicle, and an ear canal of the human for purposes of vaccination for COVID 19 disease.

12. The method of claim 11, wherein:
the source of the SARS-CoV-2 virus comprises sputum from an infected person.

13. The method of claim 11, wherein:
the lymph node is that present in the neck of the human.

14. The method of claim 11, wherein:
the vaccine composition is also introduced into a lymph node located under the armpit of the human.

15. The method of claim 11, further comprising:
administering a transfusion of blood plasma from a previously infected person to the human prior to or while introducing the vaccine composition.

16. The method of claim 15, wherein:
the transfusion is administered from 5 minutes to 1 hour prior to introducing the vaccine composition.

17. The method of claim 1,1 wherein:
the inoculation is carried out within one week from being infected with the SARS-CoV-2 virus.

18. The method of claim 11, wherein:
the vaccine composition further includes a source of influenza A virus.

19. The method of claim 18, wherein:
the influenza A virus is that containing at least one or more of the antigens h1 to h8 and n1 to n4.
